(19) United States
(12) Patent Application Publication
(10) Pub. No.: US 2010/0331949 A1
(43) Pub. Date: Dec. 30, 2010

(54) STENTS, DEVICES FOR USE WITH STENTS AND METHODS RELATING THERETO

(75) Inventor: Nagy Habib, London (GB)

Correspondence Address: OPPENHEIMER WOLFF & DONNELLY LLP, 45 SOUTH SEVENTH STREET, SUITE 3300, MINNEAPOLIS, MN, 55402, US

(73) Assignee: EMCISION LIMITED, London (GB)

(21) Appl. No.: 12/521,516

(22) PCT Filed: Jan. 11, 2008
(86) PCT No.: PCT/GB2008/000102

§ 371 Date: Sep. 15, 2010

(30) Foreign Application Priority Data
Jan. 11, 2007 (GB) 0700560.6
May 23, 2007 (GB) 0709910.4

(51) Int. Cl. A61F 2/84; A61F 2/82

(52) U.S. Cl. 623/1.11; 623/1.15

(57) ABSTRACT

A removable stent (10) has guide member pairs (16) and loops (20), the guide member pairs being used to align arms of a framework (28) which may expand or collapse the stent. The stent can be retained inside an outer sheath (30) of a catheter (32) with a collapsible filter net (34). Magnetic stents and stents with springs (170) and hinges (156) are also provided.

The present invention relates to stents, such as removeable stents. The invention also relates to stent manoeuvring devices, such as devices for moving, deploying or contracting stents in lumens or vessels of patients, or for removing or retrieving stents from such lumens or vessels. The invention also relates to methods for manoeuvring stents in such lumens and vessels and to methods for servicing such stents.

A known removeable stent is disclosed in U.S. Pat. No. 6,821,291. The stent may be removed by applying a twisting force and then an axial pulling force to the stent and such forces are not always desirable. It also appears difficult to attach a stent removal lasso to hooks of the stent. Additionally, in the prior art, different apparatus is often required for applying stents and then removing them when they need to be removed, requiring stock of multiple types of apparatus in medical facilities. Additionally, it is sometimes difficult to locate stents in the actual desired location thereof and stents often do not provide a robust structure and often have a fixed expanded diameter or cross-dimension which is not suitable for all situations.

The present invention aims to alleviate at least to a certain extent at least one of the problems of the prior art. Another aim of the invention is to provide useful stents and stent manoeuvring devices and methods.

According to a first aspect of the present invention there is provided a stent manoeuvring device comprising a catheter and connector assembly operable through the catheter, the connector assembly having at least one connector element adapted to connect to a stent and apply a substantially radially inward contraction force thereto. This has the advantage that a stent may be contracted without the need to apply twisting or axial force thereto.

The connector assembly may comprise an expandable framework and the connector element may comprise one of a plurality of elongate arms of the framework. The arms may be pivotally retained at proximal ends thereof and may have distal ends arranged to engage a stent. The framework may be collapsible to a structure in which the arms are parallel to and closely adjacent one another and may be expandable to a configuration in which the distal ends of the arms are spaced apart. This provides a relatively strong structure which can nevertheless be operated and deployed through a catheter of relatively narrow diameter if desired.

The arms may be flexible for bending upon contact with a stent. This may advantageously allow increased surface contact between the stent and manoeuvring device for greater manoeuvring control of the stent.

In the preferred embodiments, the device may include an expandable filter for catching debris. When the stent is located in a lumen or vessel of a patient containing flowable material, the filter may be located downstream of the stent in order to advantageously catch debris which may be removed from the region of the stent during stent contraction.

The device may include an outer sheath arranged to retain a contracted stent inside the device. Accordingly, a stent may be advantageously manoeuvred around inside a patient such as before stent expansion by the device, or after stent contraction, for example when the stent is being moved or fully removed from a patient.

The arms may include ratchet elements or locking elements formed thereon for locking engagement with connector portions of a stent, such as internal loop portions of a stent. Accordingly, a secure connection may be provided between the device and a stent for a high level of control during stent expansion or contraction or manoeuvring.

In some embodiments, the arms may be provided with an end stop for restricting movement between the device and a stent.

The device may include an oscillator for applying vibrational force to a stent for example an ultrasonic piezo oscillator. It is envisaged that ultrasonic cleaning of a stent to remove debris or film thereon may be provided in this way. The arms may have sharpened ends for assisting in removing built up material from the vicinity of a stent. The arms may be of electrically conducting material or may include electrical pathways for applying power, for example EM energy such as RF or microwave energy to a stent for heating thereof.

According to a further aspect of the invention there is a provided a stent servicing device comprising a catheter and an expandable framework of arms which are pivotally connected at proximal ends thereof to a central portion of the device, and which have distal ends arranged to engage a stent. The device may include a central stern and they link extending from the stent to each arm. The links may be slidable along the stem for rotating the arms. Four said arms may be provided in one embodiment, although other numbers of arms such as 3, 6 or 8 are envisaged. The links may be pivotally connected to a collar slidable along the central stem. Accordingly, a stent servicing device with a very robust yet collapsible construction may be provided. The expandable framework of arms may have an umbrella configuration.

The stent servicing device may be arranged for contacting a stent for applying RF power thereto or microwave or other electromagnetic power thereto for tissue heating purposes. The device may be arranged for manoeuvring a stent into an expanded deployed configuration thereof, or may be arranged for manoeuvring a stent into a contracted configuration thereof. The highly versatile nature of the expandable framework of arms is therefore evident.

According to a further aspect of the present invention there is provided a stent comprising a stent body, the stent body having a connector arranged for connection to a stent manoeuvring or servicing device, and a guide for guiding a stent manoeuvring or servicing device towards the connector. Accordingly, a reliable structure for connecting a stent to a stent manoeuvring or servicing device may be provided and this may be particularly useful when the stent is, for example, a removeable stent. The guide may comprise a channel. The channel may have two converging walls extending inwardly from an inner surface of the stent body.

The connector may be located inside the stent. Accordingly, a stent manoeuvring or servicing device may be advantageously located inside the stent and connected to the stent at the connector inside the stent, rather than radially outside the stent.

The stent body may be metal, for example of silver palladium. This may advantageously inhibit the build up of biofilm and debris in the region of the stent.

The stent body may include resilient struts thereon allowing expansional contraction of the stent body. The stent body may be arranged to be contractable and subsequently re-expandable. A series of stent struts may be provided spaced circumferentially around the stent body. A series of stent struts may be provided spaced axially along the stent the body.

The connector may comprise a plurality of axially spaced lugs located in alignment with the guide on the stent body. Accordingly, the guide may provide a good connection between a stent manoeuvring or servicing device and a plurality of lugs on the stent in order to provide a very secure connection therebetween for good control of the stent during manoeuvring or servicing thereof, such as expansion or contraction thereof or movement thereof within a lumen or vessel of a patient.

According to a further aspect of the present invention there is provided a magnetic stent.

Such a stent has a number of advantages concerning particularly the location and removability of a stent.

The stent may include an implantable magnetic portion arranged to be implanted in the inner wall of a lumen or vessel of a patient, and a main stent body having a magnetic body portion moveable relative to and alignable with the magnetic portion for magnetically retaining the stent body. Accordingly, the main stent body may advantageously be inserted into the implanted magnetic portion and held in position accurately by magnetic forces acting between the implantable magnetic portion and the magnetic body portion of the main stent body.

The implanted magnetic portion may comprise at least one ring such as two magnetic rings axially spaced from one another along a lumen of a patient.

The magnetic body portion may comprise at least one body ring locatable inside the at least one ring.

The stent may be provided with measuring means for measuring the position of the implantable magnetic portion relative to the main stent body. Accordingly accurate positioning of the stent may be obtained. The measuring means may include a position-sensing coil.

According to a further aspect of the invention there is provided a removable stent comprising a body formed of elongate side members, the side members being linked by connection members, the connection members enabling the body to be contracted from an expanded configuration to a contracted configuration. Accordingly a reliable structure for a stent may be provided.

The connection members may comprise articulations connected to the side members by hinges. The hinges may therefore provide the stent with great versatility, since the stent may be expanded to a multiplicity of different expanded configurations and may also be contracted. Each connection member may include two parts, the two parts being hingedly connected together. A friction damper or indexed locking system may be provided at said hinges between the two parts, for advantageously holding the stent in a selected configuration.

Each connection member may comprise a spring, the spring may advantageously be arranged to provide a selected deployment force or radially outwardly pressure when or once it is deployed into the expanded configuration of the stent.

According to a further aspect of the present invention there is provided a combination of a stent according to one of the aforementioned aspects of the invention and a stent manoeuvring or servicing device according to one of the aforementioned aspects of the invention, the device being configured to contract the stent by application of radially inward force thereto. Accordingly, the stent advantageously need not be subjected to twisting or axial forces thereon during a contraction manoeuvre. The arms of the device may be arranged to be guided by a guide of the stent to securely connect the device with a connection element of the stent. The arms of the device, where provided, may be inwardly collapsible to pull on the stent with a substantially radially inward force for contracting the stent.

According to a further aspect of the invention there is provided a method of manoeuvring a stent comprising inserting a stent manoeuvring device into a stent and contracting the stent by applying a substantially radially inward force to the stent from the stent manoeuvring device. Advantageously, the stent manoeuvring device need not apply substantial twisting or axial forces to the stent. The stent may be located, relocated, or removed in a lumen or vessel of a patient and, accordingly, undesirable forces on the lumen or vessel may be avoided.

The method may include removing the stent from a lumen or vessel of a patient.

The method may comprise contracting the stent and moving it inside a patient and expanding the stent again at a different location inside a patient.

The method may comprise passing the stent while in a contracted configuration thereof through another stent located in a lumen, such as a lumen of a patient.

The method may include expanding the stent and locating it at a position in a vessel or a lumen of a patient, and then moving the stent manoeuvring device to the other stent and contracting the other stent and then manoeuvring the other stent, such as to remove or retrieve it from a patient.

A further aspect of the invention provides a method of servicing a stent in situ in a lumen or vessel of a patient, the method comprising inserting a cleaning device into the stent and applying vibrational energy to the stent with the cleaning device to remove debris therefrom. The vibrational energy may be ultrasound, the method may include providing a filter net to catch the debris. This method may be advantageous, for example when it is desired to clean electrical contacts on a stent such as when it is desirable intermittently or periodically to apply electrical power such as electromagnetic ablation power to a stent for heating tissue in the region of a stent. However, ultrasound may be used to clean the whole stent as well as the electrical contacts.

The aforementioned aspects of the invention enable the placements of stents and allow their removal. Often, in the prior art, metal stents have been inserted and left behind but once they become blocked they can cause problems and it can be very difficult to remove them. In accordance with some at least preferred embodiments of the present invention, removable metal stents may be inserted and then subsequently removed whether they are in the heart, prostate, lungs, rectum or other vessels or lumens inside patients.

The use of an umbrella-type system in a framework of arms to unfold and open a ring-structure metal stent and subsequent removal of the umbrella is highly advantageous. Later, the open umbrella can be used to connect with particular areas in a ring-type metal stent such that the ring may collapse and the stent may be removed and a new one may replace it.

The magnetic type embodiments in which a magnetic outer ring stays definitively implanted in the patient is also highly advantageous. This type of stent may be a wall stent and the inner barrel may be changed at frequent intervals. A magnet may be used such that the barrel stays attached to the outer metal, e.g. metal mesh. Later, an operative, such as an endoscopist or interventual radiologist, can touch the barrel inner part to deactivate or reverse polarity of the magnet and enable the release of the inner side/part thereof.

The framework/umbrella system discussed above may be arranged to open and subsequently remove a filter which can be placed in the vena cava or in all types of vascular or cardiac surgery to prevent emboli moving to carotid or distal vascular structures and the filter is therefore highly advantageous.

The use of magnetic forces or power in order to align devices or provide improved touching between electrodes of stents or other medical devices contained within patients is also highly advantageous.

It is envisaged that the umbrella/framework structure/delivery tool could be used for non-balloon placement of other types of stents or metal stents to those described herein. The use of vibrational means such as ultrasound to remove biofilm or debris may also be used on other types of stents or implanted medical devices.

The present invention may be carried out in various ways and a number of preferred embodiments of stents, stent manoeuvring and servicing devices and methods in accordance with the preferred embodiments of the present invention will now be described by way of example with reference to the accompanying drawings in which:

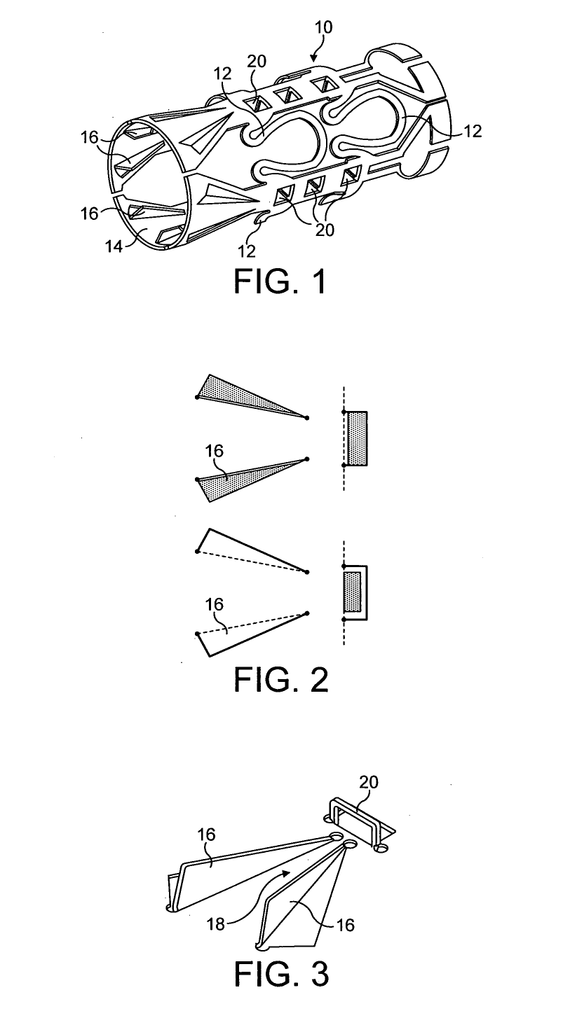

FIG. 1 is an isometric view of a preferred metal stent in accordance with an embodiment of the present invention;

FIG. 2 shows details of channels and lugs of the stent of FIG. 1;

FIG. 3 is an enlarged view of a channel/guide and connection loop of the stent of FIG. 1;

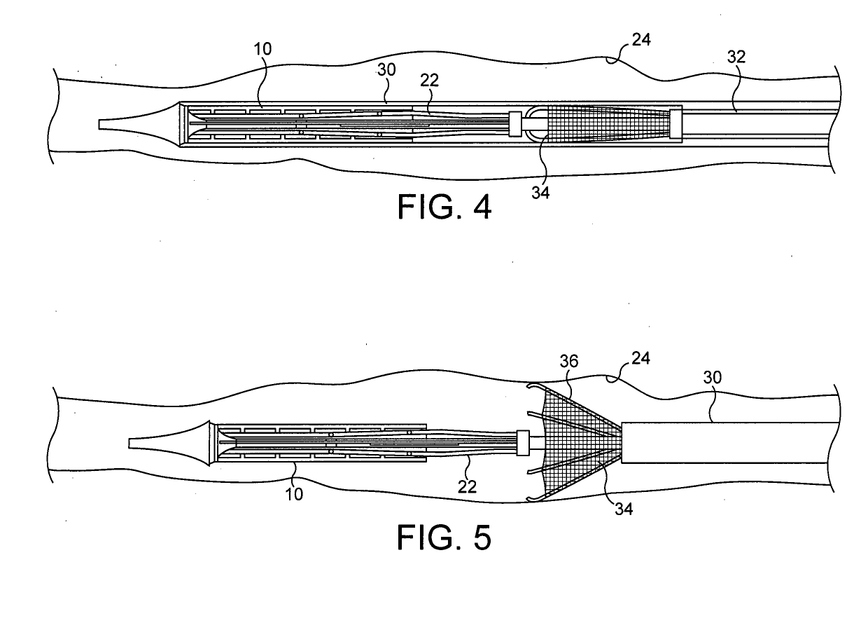

FIGS. 4 to 7 are various views of the stent of FIGS. 1 to 3 together with a preferred embodiment of a stent manoeuvring and servicing device in accordance with a preferred embodiment of the present invention;

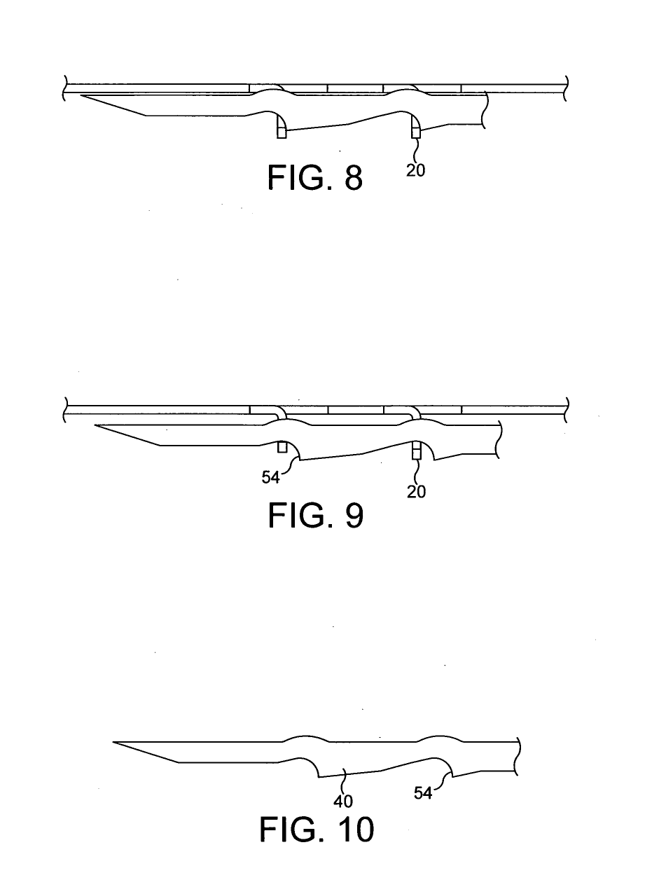

FIGS. 8, 9 and 10 shows enlarge views of arms of the stent manoeuvring and servicing device;

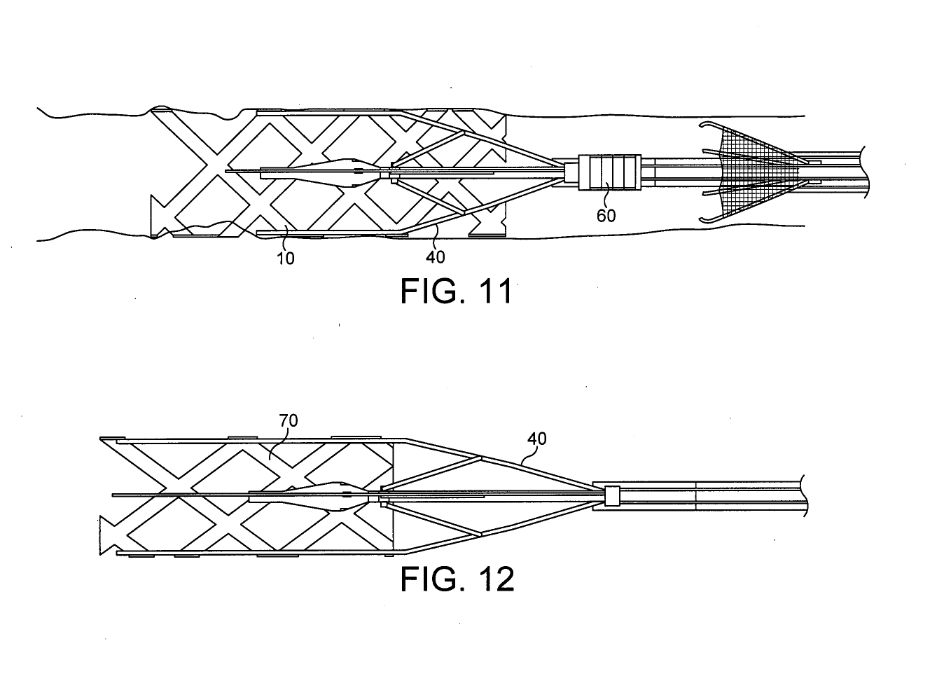

FIGS. 11, 12 and 13 shows modified versions of the devices of FIGS. 4 to 7;

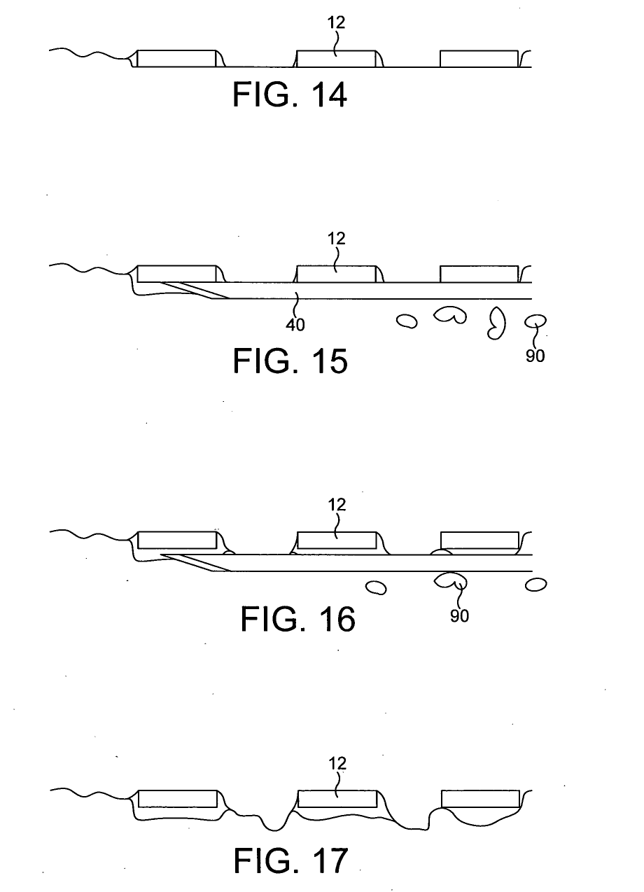

FIGS. 14, 15, 16 and 17 show views of ultrasonic cleaning for stent debris removal;

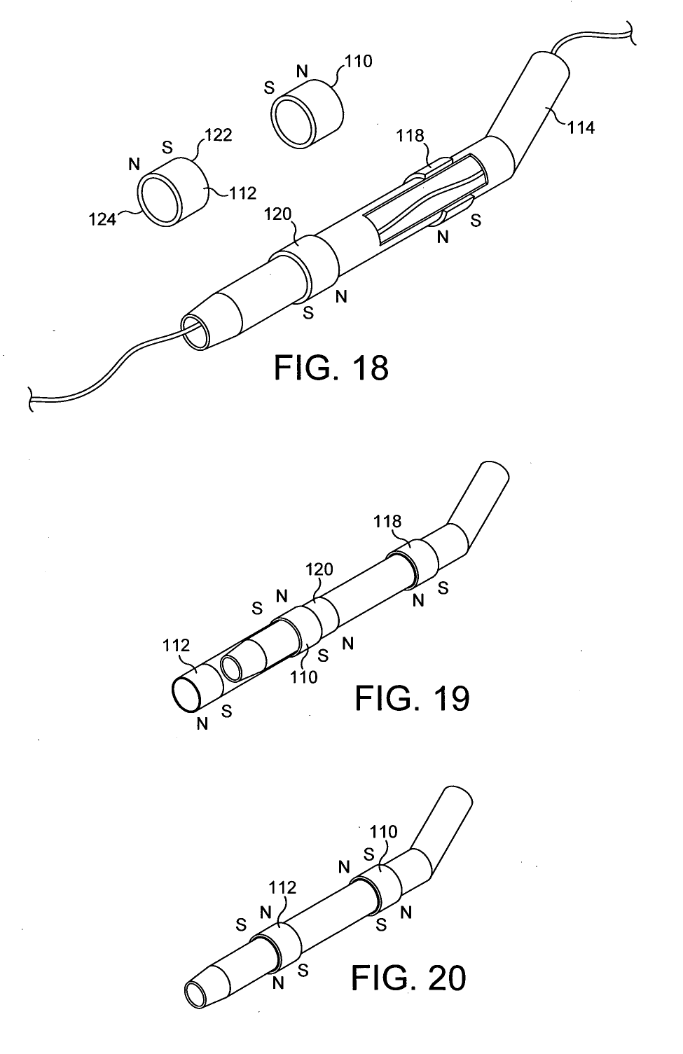

FIGS. 18 to 22 show a preferred embodiment of a magnetic step in accordance with a preferred embodiment of the present invention;

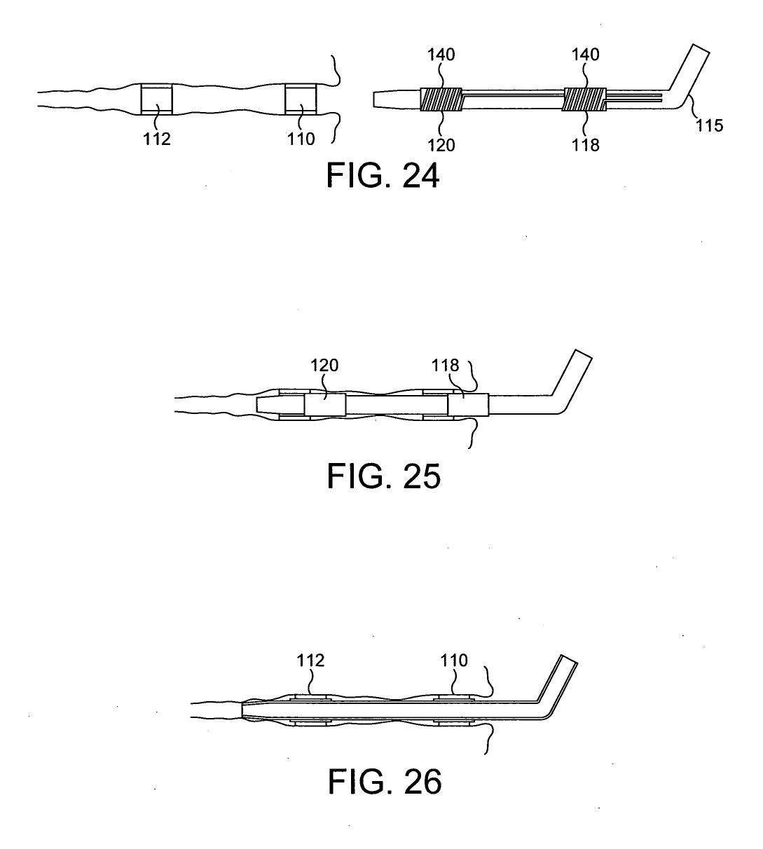

FIGS. 24, 25 and 26 show a modified embodiment of a magnetic stent;

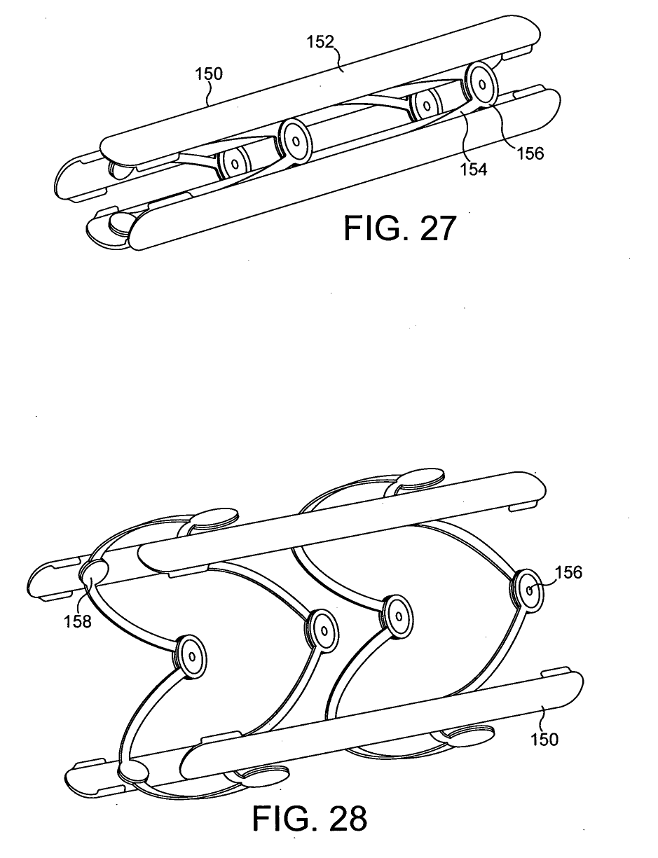

FIGS. 27 and 28 show a preferred embodiment of an articulated removable stent in accordance with a preferred embodiment of the present invention;

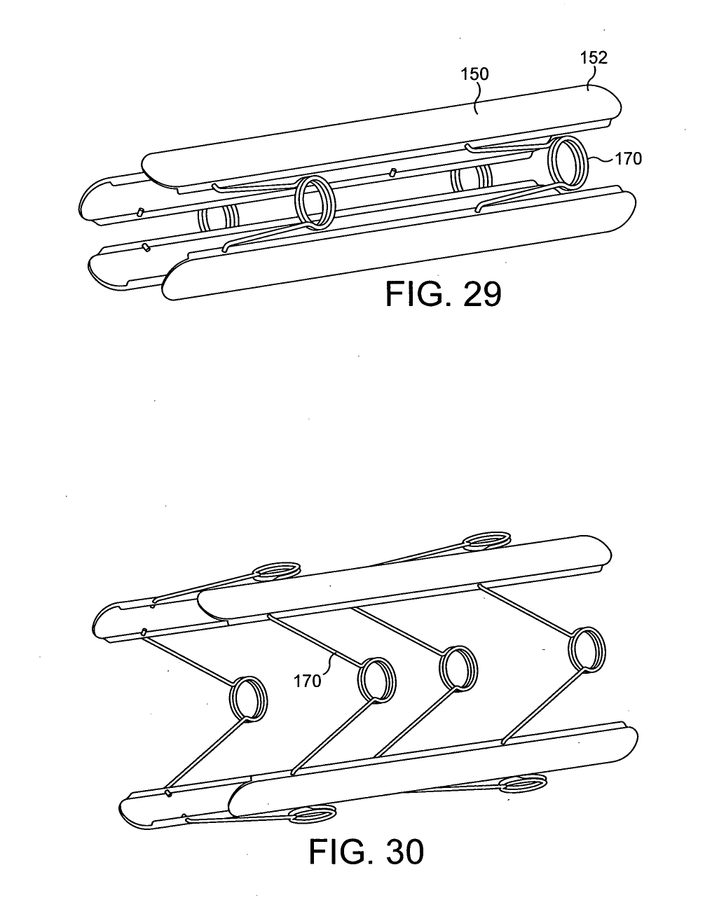

FIGS. 29 and 30 show a sprung removable stent in accordance with a preferred embodiment of the present invention; and

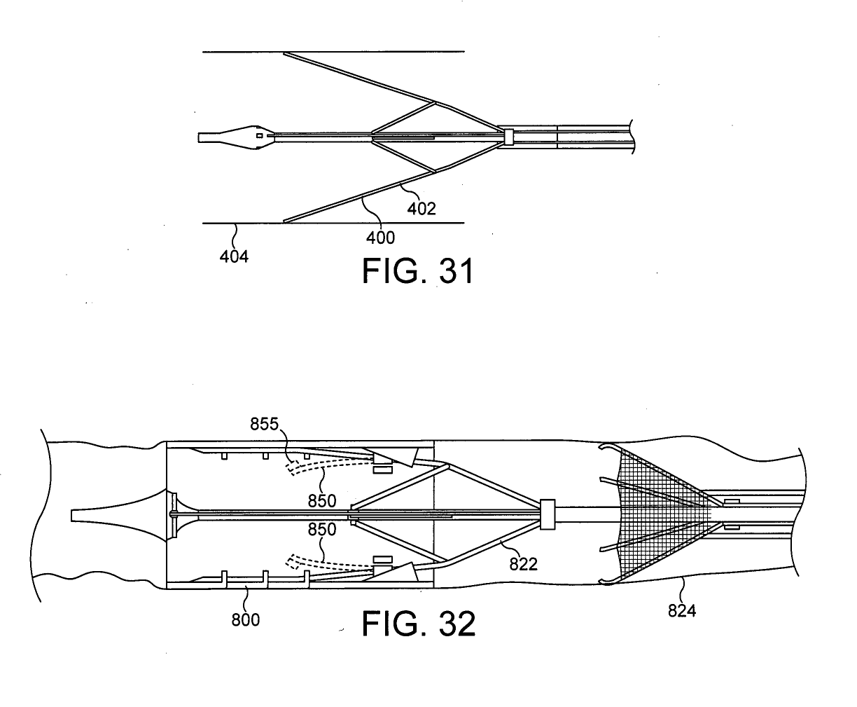

FIG. 31 shows a stent servicing device in accordance with a preferred embodiment of the present invention.

FIG. 1 shows a preferred embodiment of a metal stent (10) which is adapted for removable insertion in a lumen or stent of a patient. The stent is made of silver palladium for reduced biofilm and can also be used for heating of tissue in the region thereof, such as RF heating or other EM heating such as microwave. The stent includes resilient struts (12) which allow opening/closing or expansion/contraction of the stent with controlled force.

The stent is shown in FIG. 1 in a contracted or collapsed configuration thereof an interior surface (14) of the stent includes a series of upstanding guide member pairs (16) which form channel (18) pointing towards radially inwardly upstanding loops (20). The lugs or guide member pairs (16) are used to align arms of an umbrella framework to be described in further detail below during delivery and removal of the stent from a lumen or vessel (24) of a patient, with arms (24) of the umbrella framework (28) locating in the channels or funnels (18) and then sliding through the loops (20). The lugs and loops (16, 20) of the stent (10) may be made during laser cutting of the stent and then folded during manufacture of the stent. Alternatively they may be crimped or welded onto a mesh stent.

FIG. 4 shows the stent (10) retained inside an outer sheath (30) of a catheter (32) in a position where the catheter has been located inside the lumen (24) using a guide wire and X-ray. Also inside the sheath (30) is a collapsed filter net (34) and the collapsed umbrella framework structure (22).

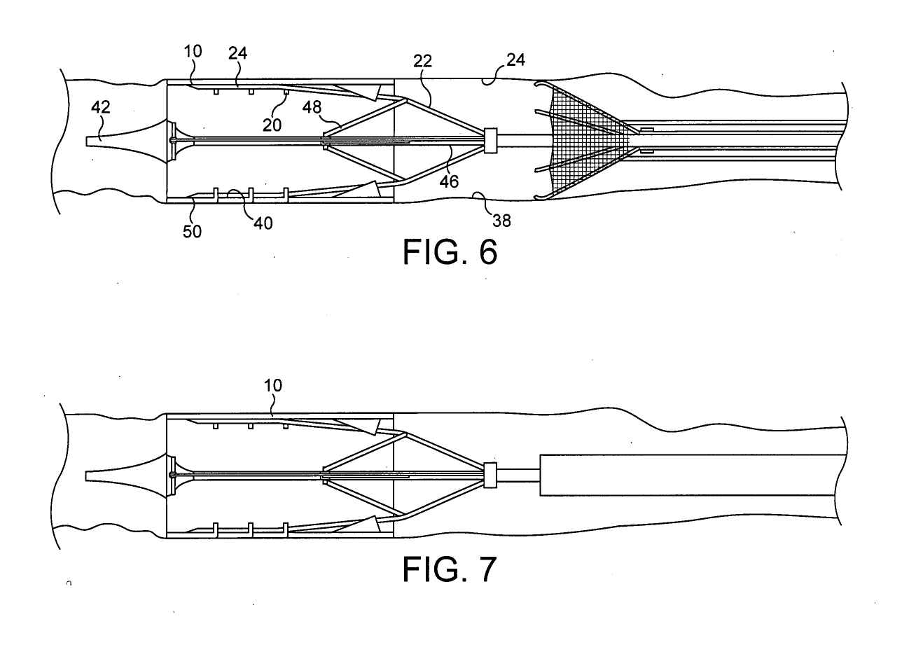

The outer sheath (30) may be pulled back to the position shown in FIG. 5 allowing the filter net (34), which is resilient, to open to a configuration in which it filters fluid flow passing along the lumen (24) or vessel. The filter net may be meshed, fiber or balloon sock with micro-holes and may have a self-expanding nitinol frame (36). In the view of FIG. 6, the stent (10) has been deployed or expanded by the framework (22) to an expanded configuration in which it engages the inner wall (38) of the lumen or vessel (24) in a configuration in which four arms (40) of the framework (22) are engaged each one through three loops (20) of the stent (10). At this stage electromagnetic power such as RF could be applied to the stent for tissue heating purposes. The catheter (32) including the frame work (22) and filter (34) and guide tip (42) may then be withdrawn from the stent by movement to the right in FIG. 6, leaving the stent in place in the lumen.

FIG. 7 is a view similar to FIG. 6, but with the filter net absent.

At a later point in time, the catheter (32) may be reinserted into the lumen and brought to a position with the tip (42) near guides (16). The sleeve (30) may then be withdrawn allowing the arms (40) to be expanded again by pulling remotely from outside the patient on inner shaft (46) so that arms (40) are moved from a substantially mutually parallel configuration by compression of linked arms (48) to bring tips (50) of the arms to engage the stent (10) between the guide lugs (16). The catheter may then be slid to the left as shown in FIG. 6 until each arm passes fully through three of the loops (20) which are aligned with the channel (18). As shown in FIG. 10, the arms (40) include ratchet or lock members (54) adapted for secure engagement in the loops (20).

The framework (22) may then be collapsed by force on the stem (46) to bring the stent back to the collapsed or contracted configuration shown in FIG. 5, so that the sleeve (30) may be slid over the stent (10) again and the stent may be moved or entirely removed from the patient. The filter net is important during the contracting operation when debris may be released from the region of the stent.

The ratchet or locking elements (54) ensure good engagement between the arms (40) and stent (10) such that the stent only moves relative to the arms (40) or detaches when required. In some embodiments, the arms (or struts) (14) may each have a single end stop and the ratchet elements (54) may be absent.

The stent may be heated using RF or ultrasonic power for tissue treatment. FIG. 11 shows a modification of the device in which an ultrasonic oscillator (60) is provided for oscillating the arms (40) against the stent (10), schematically shown in FIG. 11. The ultrasonic rubbing could also be used on other types of stent and the manoeuvring device, i.e. the components shown in FIG. 4 excluding the stent (10) may also be used for the delivery of other types of stent (70) as shown in FIG. 12.

The stent (10) shown in FIG. 1 could, instead of being made from silver palladium be coated with a silver ion-releasing coating. Either structure will advantageously reduce the biofilm build-up and reduce in turn calcium and salt build-up in the region of the stent, thereby increasing the time required between stent cleaning or removal. An alternative would be to use hydrogel or bio-glass on the stent.

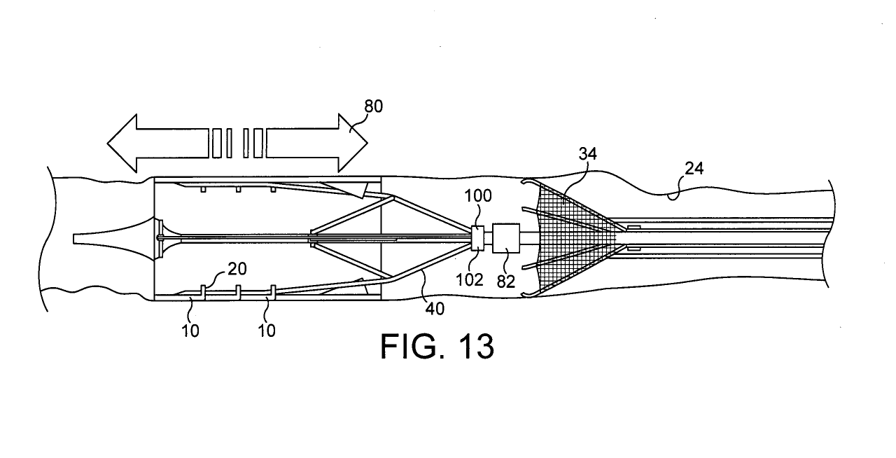

FIG. 13 shows axial rubbing of the stent (10) of FIG. 1 by the flexible arms (40), the axial oscillation caused by the ultrasound being shown by the arrow (80) and caused by ultrasound generator (82).

FIGS. 14 to 17 show stent cleaning by virtue of the ultrasound rubbing shown in FIG. 13. The ultrasound rubbing may be sufficient to locally heat tissue in the region of the stent by engagement of the arms (40) against stent struts (12). The ultrasonic rubbing may lead to debris (90) being removed and captured in the net (34), the debris consisting of materials such as biofilm, mineral, tissue and/or fatty deposits caused in stent occlusion. The ability to clean the stent struts is advantageous if RF heating is to be used since improved electrical contact through electrically conducting arms (40) to stent (10) may be achieved. The rubbing allows electrical contact between clean surfaces.

The ultrasonics could be applied via a piezo driver (100) within strut pivot (102) instead of as shown at (82), in order to oscillate struts so as to remove build-up. This may be used with conventional stents to rub away build-up and the build-up may be captured by the net filter (34). As indicated above, the RF power or other EM power for heating may be supplied to the stent (10) via the struts, the RF or other EM power source being located outside the patient and fed along to the arms (40) through the catheter. In conventional metal stents, the framework of arms (22) may be rotated axially within the stent to completely clean the stent.

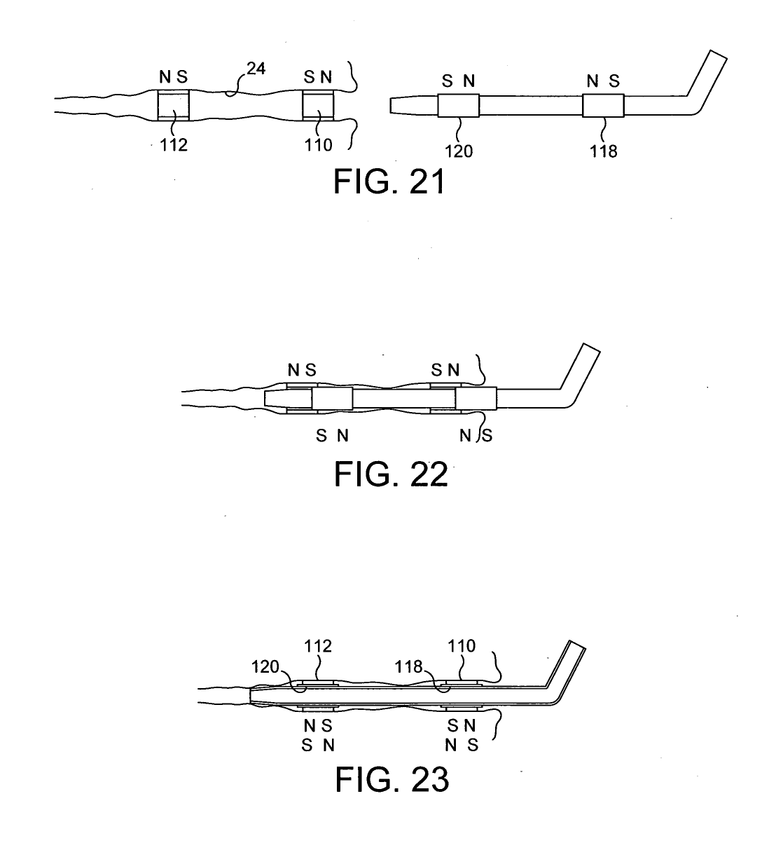

FIG. 18 shows a preferred embodiment of a magnetic stent having implantable magnetic rings (110, 112). The stent (114) is a plastic tube stent. The magnetic polarities of the implantable rings (110, 112) and corresponding magnetic rings (118, 120) on the plastic stent (114). Throughout FIGS. 18 to 23, south polarities (122) are denoted by the letter S and north polarities (124) by the letter N. FIGS. 18, 19 and 20 are isometric views showing the stent removed from, being inserted to, and magnetically held in position, and FIGS. 21, 22 and 23 are equivalent side views. The matching poles (122, 124) increase the field effect. The stent cartridge is located and held in place by the implanted magnetic rings (110, 112). The holding force of the magnetic field is greater than the flow resistance, but is such that the stent can be removed with an endoscopic retraction tool. In the configuration shown in FIGS. 19 and 22, the poles adjacent one another are the same so the stent will not rest in this configuration. However, in the configuration shown in FIGS. 20 and 23, the poles are matched and the magnetic circuit will aid the stent to locate.

The magnetic rings (110, 112) may be delivered into position in the lumen or vessel (24) by catheter. The stent (114) may be removed and replaced with another. The stent may be coated in hydrogel to both slow biofilm or other debris build-up and to act as a lubricious coating to aid the stent to self-locate as the poles (122, 124) aligned with one another. Maximum holding forces obtained once the poles are aligned as shown in FIGS. 20 and 23.

In a modification of the magnetic stent (114), coils (140) may be employed and resistance in the coils may be measured in this modified stent (115). The coils may be wrapped either around the stent or the stent magnet and use an affect similar to LVDT to aid location. Changes in the magnetic field will affect the electrical response from the coils, thereby allowing location to be known. Additionally, pole change could be used by applying voltage across the coils to repel the stent in order to aid stent removal.

FIG. 27 shows a removable stent (150) with stainless steel or plastic side bars (152) joined together by articulations (154) or tie bars (154). The tie bars are hinged at hinges (156, 158). The stent is expandable to the configuration shown in FIG. 28 and this stent (150) is scalable to cover a substantial range of application areas within patients. The stent, it is envisaged, could be heated with electromagnetic energy such as RF heating, using the umbrella framework (22) shown in FIG. 12. The framework (22) could be used for the deployment and removal of a range of different stents. If desired, the stent (150) may be deployed or expanded using a balloon system. The stent (150) may include internal loops (not shown) similar to those in the stent (10) and the umbrella system framework (22) may therefore be docked with the stent (150) and the stent may be collapsed and removed. The side bars may be opened or expanded using the same framework system.

FIGS. 29 and 30 show an alternative to pivoted tie arms using nitinol springs (170). A spring hinge is easy to manufacture at low cost for use in small lumens. The springs (170) also minimise problems of encrustation which may occur with hinges (156, 158). The springs allow for variability and the profile of the stinted area, such as if the lumen is larger at one end of the stent (150) than the other. Additionally, the springs can be tailored to apply a fixed deployment force outwardly on the lumen. The springs also allow for internal-luminal movement and may therefore be used in areas where fixed diameter stents are not suitable, or in dynamic locations.

FIG. 31 shows a stent servicing device (400) with an expandable framework (402) of arms for servicing a stent (404) by applying RF power thereto.

FIG. 32 shows a further example of a stent (800) that is shown in an expanded configuration inside a lumen (824) of a patient by a framework (822), in a similar manner to the stent shown in FIG. 7. The stent (800) includes a nitinol latch (850) which comprises at least one resilient arm and a locating lug (855) at a distal end thereof. A proximal end of the latch (800) is affixed to the stent. In use, the lug (855) of the nitinol latch locks the stent onto a catheter when the nitinol is relatively cold. However, after a heating operation the lugs (855) bend radially inwards to enable stent release from the lumen.

Various modifications may be made to the specific embodiments described without departing from the scope of the invention as defined by the accompanying claims as interpreted under Patent Law.

What is claimed:

1. A stent maneuvering device comprising a catheter and a connector assembly operable through the catheter, the connector assembly having at least one connector element adapted to connect to a stent and apply a substantially radially inward contraction force thereto.

2. The device as claimed in claim 1 in which the connector assembly comprises an expandable framework and the connector element comprises one of a plurality of elongate arms of the framework.

3. The device as claimed in claim 2 in which the arms are pivotally retained at proximal ends thereof and have distal ends arranged to engage a stent.

4. The device as claimed in claim 2 in which the arms are flexible for bending upon contact with a stent.

5. The device as claimed in claim 2 in which the aims are moveable to a contracted configuration in which they are substantially parallel.

6. The device as claimed in claim 1 further comprising an expandable filter for catching debris.

7. The device as claimed in claim 1 further comprising an outer sheath arranged to retain a contracted stent inside the device.

8. The device as claimed in claim 2 in which the arms include ratchet elements formed thereon for engagement in a connector portion of a stent.

9. The device as claimed in claim 2 in which the arms each have an end stop portion for restricting movement between the device and the stent.

10. The device as claimed in claim 1 further comprising an oscillator for applying vibrational force to the stent.

11. The device as claimed in claim 2 in which the arms have sharpened ends for removing built-up material from the vicinity of a stent.

12. The device as claimed in claim 3 in which the arms are of electrically conducting material for applying power, at an RF frequency to a stent.

13. A stent servicing device comprising a catheter and an expandable framework of arms which are pivotally connected at proximal ends thereof to a central portion of the device and which have distal ends arranged to engage a stent.

14. The device as claimed in claim 13 in which a central stem is provided and a link is provided extending from the stem to each arm.

15. The device as claimed in claim 14 in which the links are slidable along the stem for rotating the arms.

16. The device as claimed in claim 13 in which four said arms are provided.

17. The device as claimed in claim 13 which is arranged for contacting a stent to apply power thereto.

18. The device as claimed in claim 13 which is arranged for maneuvering a stent into an expanded deployed configuration thereof.

19. The device as claimed in claim 13 which is arranged for maneuvering a stent into a contracted configuration thereof.

20. A stent comprising a stent body, the stent body having a connector arranged for connection to a stent maneuvering device and a guide for guiding a stent maneuvering device towards the connector.

21. The stent as claimed in claim 20 in which the guide comprises a channel.

22. The stent as claimed in claim 21 in which the channel has two converging walls extending inwardly from an inner surface of the stent body.

23. The device as claimed in claim 22 in which the connector is located inside the stent.

24. The device as claimed in claim 20 in which the stent body is metal.

25. The device as claimed in claim 20 in which the connector and the guide are folded from the stent body which is of sheath metal.

26. The device as claimed in claim 20 in which the stent body is arranged to be contractable and then subsequently re-expandable inside a lumen or vessel of a patient.

27. The device as claimed in claim 20 in which the stent body includes resilient struts enabling expansion or contraction of the stent body.

28. The device as claimed in claim 20 in which the stent body is arranged to be contractable and then subsequently re-expandable.

29. A magnetic stent comprising an implantable magnetic portion and a main stent body having a magnetic body portion alignable with the magnetic portion for magnetically retaining the stent body.

30. (canceled)

31. The stent as claimed in claim 29 in which the magnetic portion comprises at least one ring.

32. The stent as claimed in claim 29 in which the magnetic body portion comprises at least one body ring locatable inside the at least one ring of the implantable magnetic portion.

33. The stent as claimed claim 29 and fluffier comprising measuring means for measuring the position of the implantable magnetic portion relative to the main stent body.

34. A removable stent comprising a body formed by a plurality of elongate side members, the side members being linked by connection members, the connection members enabling the body to be contracted from an expanded configuration to a contracted configuration thereof.

35. The stent as claimed in claim 34 in which the connection members comprise articulations connected to the side members by hinges.

36. The stent as claimed in claim 34 in which each connection member includes two parts, the two parts being hingedly connected together.

37. The stent as claimed in claim 34 in which each connection member comprises a spring.

38. The stent as claimed in claim 34, in which the stent body is arranged to be contractable and subsequently re-expandable inside a lumen or vessel of a patient.

39. The stent as claimed in claim 34, in which the stent body is arranged to be contractable and then subsequently re-expandable.

40. The medical assembly comprising a combination of a stent as claimed in 29 with a device as claimed in claim 1, the device being configured to contract the stent, preferably by application of substantially radially inward force thereto.

41. The medical assembly as claimed in claim 40 in which arms of the device are arranged to be guided by a guide member of the stent for connection of the device and stent together.

42. The medical assembly as claimed in claim 40 in which arms of the device are inwardly collapsible to pull substantially radially inwardly upon a connector of the stent in order to contract the stent to a contracted configuration thereof.

43. A method of maneuvering a stent comprising inserting a stent maneuvering device into a stent and contracting the stent by applying a substantially radially inward force to the stent from the stent maneuvering device.

44. The method as claimed in claim 43 which includes removing the stent from a lumen or vessel of a patient.

45. The method as claimed in claim 43 which comprises contracting the stent and moving it inside a patient.

46. The method as claimed in claim 43 which comprises passing the stent while in a contracted configuration thereof through another stent located in a lumen of a patient.

47. The method as claimed in claim 46 which includes expanding the stent and locating it at a position in a lumen or vessel of a patient and then moving the stent maneuvering device to the other stent and contracting the other stent and moving it inside the patient using the stent maneuvering device.

48. A method of servicing a stent in situ in a lumen or vessel of a patient, the method comprising inserting a servicing device into a stent and carrying out a servicing operation upon the stent.

49. The method of servicing a stent as claimed in claim 48 which includes inserting a cleaning device into the stent and applying vibrational energy to the stent with the cleaning device to remove debris therefrom.

50. The method as claimed in claim 48 in which the vibrational energy is ultrasound.

51. The method as claimed in claim 49 further including providing a filter net to catch the debris.
